(19) United States
(12) Patent Application Publication
(10) Pub. No.: US 2022/0057313 A1
(43) Pub. Date: Feb. 24, 2022

(54) Diffusion Cell Clamping and Assembly Tools

(71) Applicants: Joseph Zhili Huang, Monroe, NJ (US); Ping Wen, Bartlett, TN (US)

(72) Inventors: Joseph Zhili Huang, Monroe, NJ (US); Ping Wen, Bartlett, TN (US)

(21) Appl. No.: 16/998,475

(22) Filed: Aug. 20, 2020

(51) Int. Cl. G01N 13/00; G01N 33/15; B01L 9/00

(52) U.S. Cl. CPC G01N 13/00; G01N 33/15; G01N 2013/003; B01L 2200/14; B01L 2200/087; B01L 9/50

(57) ABSTRACT

A diffusion/permeation cell, commonly referred to as a Franz cell, is provided for topical or transdermal drug delivery research and development in the pharmaceutical industry. The cell comprises a receptor container, a donor chamber, a quick clamping apparatus, and/or an assembly tool. Systems and methods provide a cost-effective diffusion cell, especially for use with an automatic diffusion release testing system.

This application claims the benefit of U.S. Provisional Application No. US 62/890,713, filed on Aug. 23, 2019.

### FIELD OF THE INVENTION

1. The present disclosure is in the field of apparatus for topical or transdermal diffusion or permeation testing. More particularly, the present disclosure provides systems and methods of a Franz Cell with clamping apparatus and assembly tool for drug diffusion or permeation testing to determine the transfer of an ingredient through a membrane in drug delivery development.

### BACKGROUND OF THE INVENTION

2. Diffusion testing is a release rate test of an active pharmaceutical ingredient in semisolid form as it permeates through a skin-like membrane into a solution. Diffusion replicates the process of skin-applied medicine as it permeates the skin into the body for local or systemic action.

3. Diffusion testing may be ideal for quality control of topical preparations. Diffusion testing measures a rate that an active pharmaceutical ingredient is released from a semisolid preparation, providing quality control analysts with critical performance data.

4. The use of in vitro release tests (IVRT) to evaluate drug release from semi-solid formulations has become routine for topical product development. Like dissolution testing for solid dosage forms, IVRT for semi-solid dosage has become increasingly important.

5. As recited by FDA Guidance, “In vitro release is one of several standard methods that can be used to characterize performance characteristics of a finished topical dosage form (i.e., semi-solids like creams, gels, and ointments).” IVRT has shown promise as a means to comprehensively study continuous delivery of active components from semi-solid products.

6. A common IVRT method employs an open chamber design such as the Franz diffusion cell system, which comprises a donor chamber on its top, a receptor chamber below, and a membrane that separates them. The membrane can be a synthetic membrane, a tissue construct, or biological sample, such as cadaver skin. The donor chamber contains the test drug product while the receptor chamber is filled with collection medium.

7. Diffusion of the drug from the semisolid product across the membrane is monitored by assay of sequentially collected samples of the receptor medium. At predetermined time points, an aliquot of medium is sampled from the receptor chamber for drug content analysis, usually by HPLC. The receptor chamber is topped off with fresh medium after each sampling.

8. A traditional diffusion testing system having a group of at least six cells with a magnetic bar drive to control the mixing of each cell receptor chamber, and a circulating bath providing heated water to the jacketed cells to maintain a constant temperature may be a desirable diffusion system. This system may provide flexibility in designing method according to need and produce reproducible release profiles for a particular drug molecule. The automation of such traditional systems only for sampling and filling operation may result in simpler, easy to use and cost-effective diffusion systems.

9. Problems encountered during use of the diffusion cell system are:
1) Assembly of the donor chamber and the receptor chamber together with the membrane is time-consuming and strenuous. The apparatus to be used to hold the two chambers together is either a pair of pincers or a pair of stainless steel plate with four screws. The clamping force to be applied against the donor chamber and the receptor holding them with the membrane in position is not uniform. Leakage of the medium from the receptor chamber or shift of the donor chamber and the receptor chamber may occur frequently.
2) Due to the time consumed in the assembly of the diffusion cells, there is time lag between the first and the last of the cell group, which may generate divergence of the testing results from the first and the last diffusion cell.
3) The current clamping apparatus is difficult to use with automated diffusion testing systems for operation of shaking, tilting, or refilling. During these operations, the diffusion cell needs to be held firmly in position.

10. Based therefore on analyses of problems associated with the use of current diffusion cell systems, there exists a need to develop a diffusion cell with more simple design to address at least the issues described above.

### SUMMARY

11. The present disclosure provides systems and methods of a diffusion cell with an adaptive clamping system. A simple, efficient, easy to use, and cost-effective diffusion cell to be used with an automatic diffusion release testing system is provided herein.

12. In a first embodiment, a diffusion cell is provided comprising a receptor container, a donor chamber, and a quick clamping apparatus. The apparatus comprises a clamping nut, a donor cap, and a pressing screw. The diffusion cell is assembled for transdermal drug diffusion testing.

13. In a second embodiment, an assembly tool may be used for quickly and conveniently putting the diffusion cell together. The assembly tool may comprise structures that take the contour profiles of the diffusion cell.

14. The first and the second embodiments summarized above are intended to be non-limiting as regards use of the components and interactions described herein. Modifications may be made to the components and interactions provided that do not depart from the present invention as described herein.

### BRIEF DESCRIPTION OF THE FIGS.

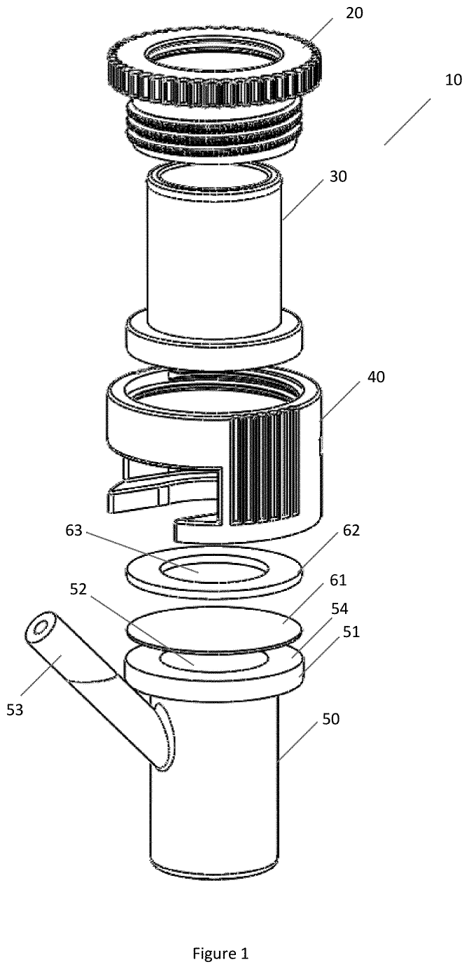

15. FIG. 1 is an exploded perspective view of an embodiment of a diffusion cell together with a receptor container, a donor chamber, and a quick clamping apparatus;

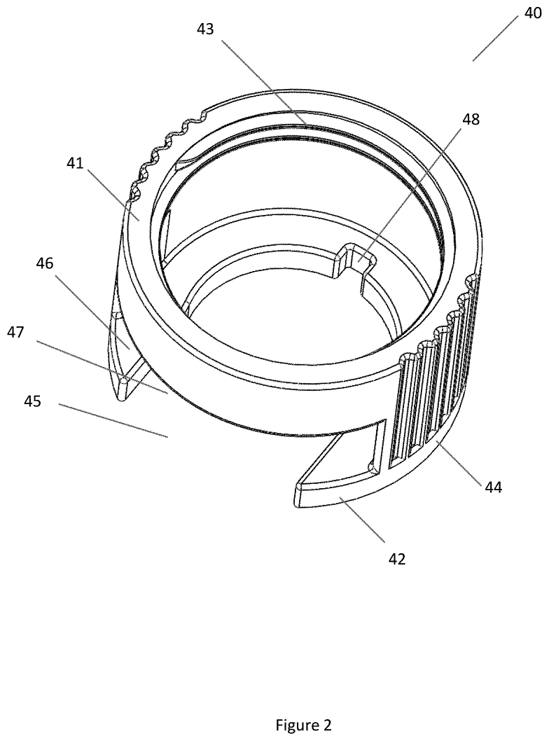

16. FIG. 2 is a perspective view of the clamping part 40 depicted in FIG. 1;

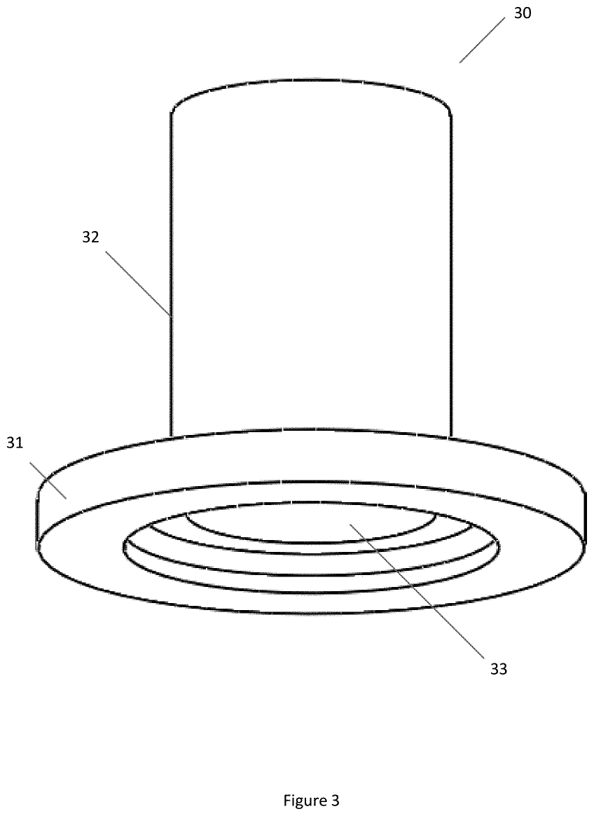

17. FIG. 3 is a perspective view of the pressing part 30 depicted in FIG. 1;

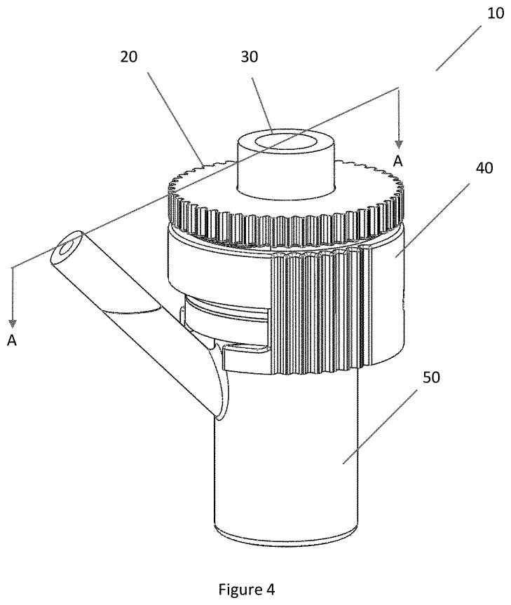

18. FIG. 4 is a perspective view of an assembled diffusion cell with the clamping system provided herein with a membrane and a drug solution holder;

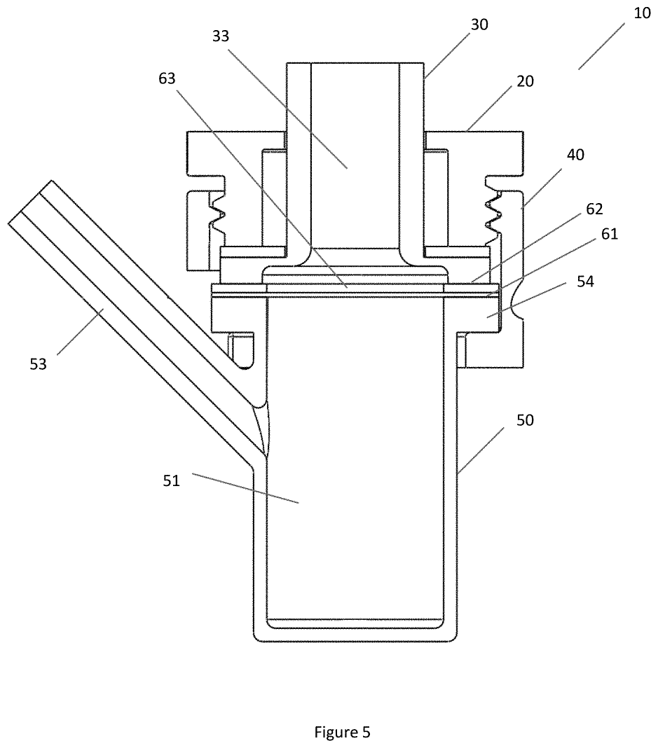

19. FIG. 5 is an A-A sectional view of the assembled diffusion cell with a membrane and a drug solution holder depicted in FIG. 4;

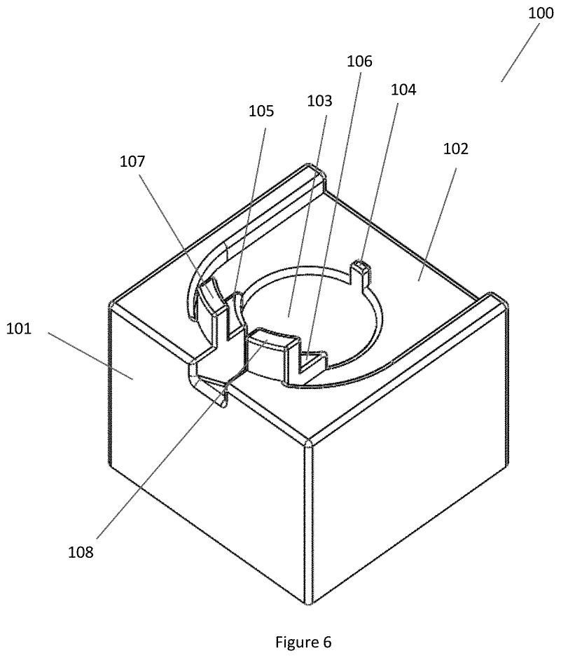

20. FIG. 6 is a perspective view of an embodiment of an assembly tool for the diffusion cell shown in FIGS. 1-5;

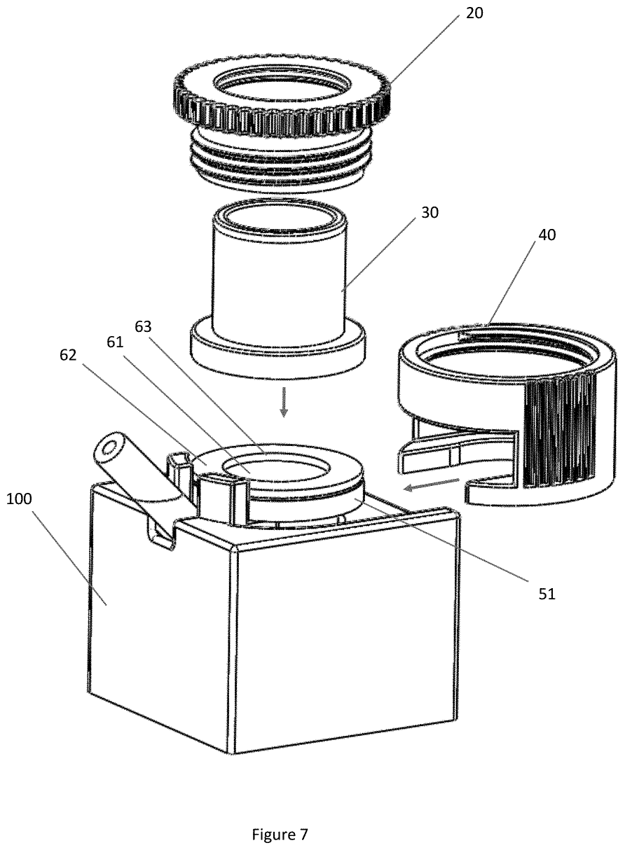

21. FIG. 7 illustrates an assembly procedure of the diffusion cell with the assembly tool in FIG. 6.

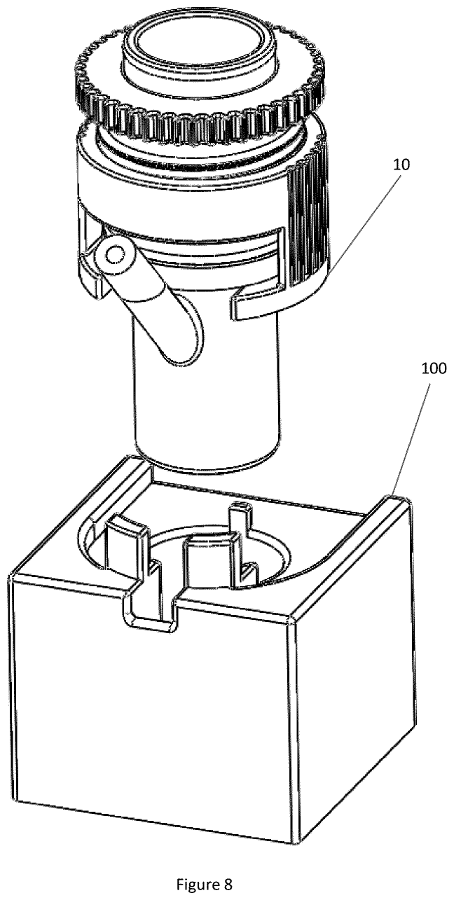

22. FIG. 8 illustrates the assembled diffusion cell releasing from the top of the assembly tool.

### DETAILED DESCRIPTION

23. Referring the figures, a diffusion cell 10 is provided. The diffusion cell 10 includes a thin walled receptor container 50, which is normally constructed of glass with a receptor fluid chamber 52, an annular flange 51, and a fluid sampling and refilling tube 53. The receptor fluid chamber 52 has a closed bottom and an open top.

24. The annular flange 51 at the upper portion of the receptor container 50 is expanded to be of a greater diameter than the lower portion of the chamber 52. A flat surface 54 is at the top of the annular flange 51. The receptor fluid chamber 52 is filled with receptor fluid that may be water, saline or other liquids.

25. The receptor fluid may be sampled and refilled by means of the sampling and refilling tube 53. Normally, a syringe pump with a long needle is used to perform the sampling and refilling the receptor fluid. The long needle with smaller diameter than the tube size of the sampling and refilling tube 53 is inserted into the receptor fluid chamber 52. The opening of the tube 53 is higher than the flat surface 54 of the receptor fluid chamber 52.

26. A membrane 61 is placed on the flat surface 54. The function of the membrane 61 is to essentially simulate human skin or animal skin and form the closed receptor fluid chamber 52 that is the body under the skin. The membrane 61 could be a cadaver skin or could be a synthetic material that essentially simulates human skin. On the membrane 61 is located a donor plate 62.

27. The donor plate 62 is in the shape of a washer of a certain thickness with a center opening that is as same as the opening of the receptor fluid chamber 52 in diameter. Typically, the donor plate 62 is constructed of silicone rubber, but other materials such as glass, teflon, or plastic can be used. The opening of the donor plate 62 together with the membrane 61 forms a donor chamber 63. A donor material that is in the form of an ointment, a cream, or a semi-solid material such as a gel, is filled the donor chamber 63.

28. On the top of the donor plate 62, a donor cap 30 is located. The donor cap 30 comprises an annular flange 31, a central opening 33, and a tubular structure 32. The annular flange 31 is as same as the annular flange 51 on the receptor fluid chamber 52 in outer side diameter. The center opening is also as same as the center opening of the donor plate 62 in diameter. The tubular structure 32 is designed for observation of donor material in the donor chamber and refilling of donor material. The donor cap 30 is made of inert material such glass or teflon.

29. It may be desirable and necessary for the donor plate 62 and the membrane 61 to be tightly restrained in position between the annular flange 31 of the donor cap 30 and the annular flange 51 of the receptor fluid container 50. To achieve this, a quick clamping apparatus is used, which is constructed by a clamping nut 40 and a pressing screw 20.

30. The clamping nut 40 comprises a body 41, a U-shaped bottom plate 42, a side opening 45 and 47, and threads 43 at its upper body. The U-shaped bottom plate 42 has the lower opening 45 with the dimension equal to the diameter of the lower portion of the receptor fluid container 50, and upper opening 47 with the dimension equal to the diameter of the annular flange 51.

31. The annular flange 51 of the receptor fluid container 50 can be inserted into the clamping nut 40 through the side opening 45 and 47. The bottom surface of the annular flange 51 attached to the top surface 46 of the U-shaped bottom plate 42.

32. The pressing screw 20 comprises an opening 21 and threads 22. The opening 21 allows the tubular structure 32 of the donor cap 30 to be put through. The threads 22 is same as the threads 43 on the clamping nut 40.

33. For assembly of the diffusion cell, as the first embodiment of this invention, the following steps are involved:
1) The donor plate 62 is placed on the top of the membrane 61.
2) The donor material in a form of an ointment, a cream, or a semi-solid, is filled the donor chamber 63.
3) The donor plate 62, the membrane 61, and the donor material filled in the donor chamber are placed on the top surface 54 of the receptor fluid container 50. Together they are inserted into the clamping nut 40 through its side opening 45 and 47. The bottom surface of the annular flange 51 is attached to the top surface 46 of the U-shaped bottom plate 42.
4) The donor cap 30 is placed on the top of the donor plate 62 through the threads 43 opening of the clamping nut 40.
5) The pressing screw 20 is screwed down to the clamping nut 40, which presses on the donor cap 30, so that the diffusion cell is assembled.

34. The donor cap 30, the donor plate 62, and the membrane 61 are fastened to the top flat surface 54 of the receptor container 50 through the quick clamping apparatus. The donor material is in contact with the upper surface of the membrane 61. The lower surface of the membrane 61 is in continuous contact with the receptor liquid in the receptor fluid chamber 52. It can thus be tested how the active ingredients of the donor material penetrates the membrane 61 into the receptor fluid chamber 52, through sampling the receptor liquid.

35. The disassembly of the diffusion cell may be accomplished as follows.

The pressing screw 20 is unscrewed from the clamping nut 40. The donor cap 30, the donor plate 62, and the membrane 61 are released from the top flat surface 54 of the receptor container 50. The receptor container 50 can be then slid out from the opening 45 and 47 of the clamping nut 40.

36. The advantages of this embodiment of the diffusion cell with the quick clamping apparatus are:
1) the pressing screw 20, the donor cap 30, the donor plate 62, the receptor container 50 and the clamping nut 40 are concentric. When they are assembled together, the donor chamber 63 is aligned with the orifice of the receptor fluid chamber 52;
2) mechanism can be designed on the body of the clamping nut 40 or the donor cap 30 for holding the diffusion cell in place for automatic operation;
3) the quick clamping apparatus is compact such that after the assembly with the diffusion cell, any effects from shanking, moving, or tilting do not bear on the test results.

37. For further improving the operation of the assembly of the diffusion cell 10, FIG. 6 to FIG. 8 show a further embodiment of this invention, an assembly tool 100. The assembly tool 100 consists of a body 101 with top surface 102 and recess 103, raised pallets 104, 105 and 106, and baffle plates 107 and 108. The recess 103 takes the contour profile of the receptor container 50, which accommodates the receptor container 50.

38. The top surface 102 takes the U-shape contour profile of the clamping nut 40. The raised pallets 104, 105 and 106 support the annular flange 51 of the receptor container 50. On the bottom plate 42 of the clamping nut 40 there is a recess 48 to fit the raised pallet 104. The baffle plates 107 and 108 are used to prevent the shifting or incorrect positioning of the donor plate 62 and the membrane 61 during the assembly.

39. To assemble the diffusion cell on the assembly tool, as the second embodiment of this invention, the following steps are involved:
1) The receptor container 50 is placed into the recess 103, its annular flange 51 is seated on the top of the raised pallets 104, 105 and 106.
2) The refilled the donor chamber 63 of the donor plate 62 together with the membrane 61 is put on the top surface 54 of the receptor fluid container 50 with the guidance of the baffle plates 107 and 108.
3) The clamping nut 40 is inserted onto the assembly tool with the receptor fluid container 50, through the U-Shape bottom plate 42 touching the top surface 102, and the top surface 46 of the U-shaped bottom plate 42 attaching the bottom surface of the annular flange 51.
4) The donor cap 30 is pressed down on the top of the donor plate 62 through the threads 43 opening of the clamping nut 40.
5) The pressing screw 20 is screwed down to the clamping nut 40, which presses on the donor cap 30.
6) The diffusion cell is and can be released from the top of the assembly tool 100, as shown in FIG. 8.

40. The advantages of this embodiment of the diffusion cell assembly tool are: the assembly tool 100 helps the receptor container 50 in place and the diffusion cell 10 can be assembled together in a prompt fashion. The possible errors caused by incorrect positioning and shifting can be avoided.

41. Although the present disclosure has been provided with reference to the particular embodiments described herein, it is to be understood that these embodiments are merely illustrative of certain principles and applications of the present invention. Numerous modifications may be made to the illustrative embodiments and other arrangements may be devised without departing from the spirit and scope of the present invention as defined by the claims.

What is claimed is:

1. A diffusion cell, comprising:
a receptor container,
a donor chamber, and
a quick clamping apparatus, comprising:
a clamping nut,
a donor cap, and
a pressing screw,
wherein the diffusion cell is directed to at least one of topical and transdermal drug diffusion testing.

2. The diffusion cell of claim 1, wherein the clamping nut has a U-shape bottom plate and the threads on its upper body.

3. The clamping nut of claim 1, wherein the preferred material for the clamping nut is plastics and made by 3D printing or injection model fabrication.

4. The clamping nut of claim 1, wherein the donor cap is made from inert material comprising at least one of glass and teflon.

5. The diffusion cell of claim 1, wherein the pressing screw has an opening at its center and the threads on its body, wherein a preferred material is plastics and made by 3D printing or injection model fabrication.

6. A diffusion cell assembly tool, comprising:
a body;
its top surface;
a recess;
raised pallets; and
baffle plates.

7. The diffusion cell assembly tool of claim 6, wherein the top surface has a contour profile of the U-shape bottom plate of the clamping nut.

8. The diffusion cell assembly tool of claim 6, wherein the recess has a contour profile of the receptor container of the diffusion cell.

9. The diffusion cell assembly tool of claim 6, wherein a height of the raised pallets is equal to a height of the U-shape bottom plate of the clamping nut.

10. The diffusion cell assembly tool of claim 6, wherein the baffle plates are higher than the thickness of the annular flange of the receptor container.

11. The diffusion cell assembly tool of claim 6, wherein the body of the tool is made from 3D printing or plastic injection model.

12. A method of assembling a diffusion cell, comprising:
placing a donor plate with a center opening atop a membrane to form a donor chamber;
filling the donor chamber with donor material;
placing the donor plate, the membrane, and the donor material on a top surface of a receptor fluid container;
inserting the donor plate, the membrane, the donor material, and the top surface through a side opening of a clamping nut;
placing a donor cap on a top of the donor plate through a threaded opening of the clamping nut; and
screwing a pressing screw down to the clamping nut.

13. The method of claim 12, wherein the screwing action results in pressure on the donor cap.

14. The method of claim 12, wherein the donor cap, the donor plate, and the membrane are fastened to the top flat surface of the receptor container through a quick clamping apparatus.

15. The method of claim 12, wherein the donor material is in contact with an upper surface of the membrane.

16. The method of claim 12, wherein a lower surface of the membrane is in continuous contact with the receptor liquid in the receptor fluid chamber.

17. The method of claim 16, wherein the continuous contact promotes testing of penetration by active ingredients of the donor material through the membrane into the receptor fluid chamber.

18. The method of claim 17, wherein the testing is conducted through sampling the receptor liquid.

19. The method of claim 12, further comprising the donor cap is made from inert material comprising at least one of glass and teflon.

20. The method of claim 12, further comprising the pressing screw has an opening at its center and the threads on its body, wherein the screw is formed via plastic injection molding methods.
